(19) United States
(12) Patent Application Publication
(10) Pub. No.: US 2008/0228209 A1
(43) Pub. Date: Sep. 18, 2008

(54) SYSTEM AND METHOD FOR REMOVAL OF MATERIAL FROM A BLOOD VESSEL USING A SMALL DIAMETER CATHETER

(76) Inventors: Richard M. DeMello, Stow, MA (US); Jonathan R. DeMello, Stow, MA (US); Richard R. Heuser, Phoenix, AZ (US); Maureen A. Finlayson, Stow, MA (US); Jon Burkhardt, Ashland, MA (US); Craig Parker, Pelham, NH (US)

Correspondence Address: CESARI AND MCKENNA, LLP, 88 BLACK FALCON AVENUE, BOSTON, MA, 02210, US

(21) Appl. No.: 12/098,201

(22) Filed: Apr. 4, 2008

Related U.S. Application Data
(63) Continuation-in-part of application No. 11/583,873, filed on Oct. 19, 2006.
Application No. 11/583,873 is a continuation-in-part of application No. 11/074,827, filed on Mar. 7, 2005.
(60) Provisional application No. 60/551,313, filed on Mar. 8, 2004.

(51) Int. Cl. A61B 17/22

(52) U.S. Cl. 606/159

(57) ABSTRACT

This invention provides a small diameter snare device and device for thrombus removal consisting of a hollow, elongate, thin-walled outer sheath. A single central core wire extends through the entire length of the sheath. The outer diameter of the core wire is sized close to the inner diameter of the sheath while allowing for axial sliding, in order to maximize the support to the body portion of the snare device. A tool tip or “capture segment” at the distal end of the sheath and core wire can be controllably expanded to engage a thrombus and remove the thrombus from the blood vessel.

### CROSS-REFERENCE TO RELATED APPLICATIONS

The present application is a continuation-in-part of commonly assigned copending U.S. patent application Ser. No. 11/583,873, which was filed on Oct. 19, 2006, by Jonathan R. DeMello, et al. for a SYSTEM AND METHOD FOR REMOVAL OF MATERIAL FROM A BLOOD VESSEL USING A SMALL DIAMETER CATHETER, which is a continuation-in-part of U.S. patent application Ser. No. 11/074,827, which was filed on Mar. 7, 2005, by Richard M. DeMello, et al. for a SMALL DIAMETER SNARE, which claims the benefit of U.S. Provisional Patent Application Ser. No. 60/551,313, which was filed on Mar. 8, 2004, by Richard M. DeMello et al., for a SMALL-DIAMETER SNARE, each of which are hereby incorporated by reference.

### BACKGROUND OF THE INVENTION

1. Field of the Invention

The present invention relates to surgical catheters, and more particularly to devices for removing thrombus, and other blockages and materials within blood vessels.

2. Background Information

Certain snare and similar devices have become available over recent years for retrieving malfunctioning or misplaced devices or blockages such as plaque and thrombus within the cardiovascular and non-vascular regions of the body. These typically consist of fairly large diameter sheaths, which house a movable central wire or wires whose distal ends are formed into a loop, plurality of loops or other purpose-built shape. The loop is used to ensnare and capture the desired object for withdrawal and removal from the body, while other shapes may be used to grasp or capture softer biological materials. In use, the snare or another distal tool is typically passed through a guiding catheter or other introducing catheter that is placed within the vasculature and is directed to the vessel or area where the misplaced or malfunctioning device is located. The snare/distal tool can then capture the intended device or material and retrieve it out of the body through the introducing catheter or by withdrawing both the snare and the introducing catheter in tandem.

Currently available snares and similar distal tools are designed using large diameter outer sheaths that require larger entry sites. This may result in complications such as excessive bleeding and/or hematomas. Additionally, because of the large diameter, it may be necessary to remove the existing catheters and exchange to other larger devices increasing the overall time and cost of the procedure. A third disadvantage of the old means is that the outer sheath, which is typically made of a plastic material, exhibits little or no torque control, which can make ensnaring the misplaced or malfunctioned device or removing other materials very difficult. Lastly, because of the size and stiff design of these snare/distal tool devices, they have a very sharp distal leading edge which cannot be safely advanced into small diameter vessels such as those in the coronary and cerebral vasculature without risking damage to the vessel wall. An exemplary small-diameter snare design that satisfies many of the concerns above is provided in commonly owned U.S. Pat. No. 6,554,842, entitled SMALL DIAMETER SNARE by Heuser, et al., the teachings of which are expressly incorporated herein by reference.

Devices, such as the exemplary Heuser design, are characterized by a small-diameter outer sheath that has a relatively thin wall (for example, approximately 0.0020 inch or less in wall thickness) so as to accommodate an axially movable/rotatable central core wire of approximately 0.008 inch. The structure allows a snare loop attached to the distal end of the core wire and housed within the open distal end of the sheath to be selectively extended from the sheath end, withdrawn and torqued. This sheath is at least partially composed of metal. The thinness of the tube, and its metallic content make it susceptible to splitting, fracturing and fatigue failure under stress. In addition, the metal section of the tubular outer sheath tends to experience permanent (plastic) deformation when bent, and once deformed, the central core wire will tend to bind upon the lumen of the sheath, rendering the device inoperable for its intended purpose. In addition, the outer wall of the metal tube section has a lubricious coating, such as PTFE (Teflon), which is typically approximately 0.0010 inch in thickness. This necessitates further downsizing of the sheath overall outer diameter thereby reducing the inner diameter available for accommodating the central core wire, thereby further increasing the risk of inadvertent failure of the device through breakage or plastic deformation.

Further considerations arise in the case of a non-snare device used to remove materials from blood vessels. Within the U.S. alone, approximately 700,000 strokes occur every year. The majority of these (83%) are ischemic strokes due to blood clots (thrombus) that become lodged in and block cerebral vessels. It has been documented that if the blockage can be eliminated within a short period of time (up to 8 hours), the patient can experience a full recovery from the stroke. Presently, clot-dissolving drugs can be administered to break up the clot and restore blood flow, however these drugs must be administered within 3 hours of symptom onset as they take considerable time to become effective. Unfortunately, not all patients are medically eligible to receive these drugs and most frequently patients, do not arrive for medical treatment within the 3 hour limit. In these patients, mechanical removal of the blood clot has been shown to have a significant positive outcome.

Several devices have been designed to break up and suction-out thrombus in the large vessels of the legs and coronary arteries. These use a variety of means to accomplish this such as water jets, mechanical maceration, ultrasound or photo-acoustic shock waves, and laser ablation. All of these devices however, have limitations when working in the cerebral vessels. First they tend to be large and bulky and very difficult or impossible to navigate above the skull base and secondly, their therapeutic means can be extremely vigorous resulting in damage to the delicate blood vessels in the brain. They also require removal of an already placed microcatheter from the patient in exchange for their device.

One device used to treat blood clots is a mechanical capture device whereby the blood clot is grasped and pulled out of the distal vessels of the brain. The MERCI retrieval device (available from Concentric Medical of Mountain View, Calif.) is a 0.014-inch guidewire that can be passed into the blood clot as a straight wire and then can be remotely shaped into a corkscrew configuration, becoming intertwined within the blood clot. The wire is then withdrawn from the distal cerebral vessel pulling the blood clot with it. Although this device addresses the ability to navigate above the skull base, it has one major shortcoming. That is the corkscrew segment of the wire must be very soft and flexible in order to navigate within the brain. This reduces the ability of the device to remain in the corkscrew shape as it is withdrawing the blood clot. During withdrawal, the wire can straighten and the blood clot can be partially or fully released resulting in greater injury to the patient through thromboembolism. A more effective tool for removal of thrombus reduced risk of release or breakup and the ability to navigate smaller blood vessels is highly desirable.

### SUMMARY OF THE INVENTION

This invention overcomes prior disadvantages by providing a small-diameter snare device and a device for removing thrombus and other materials from vascular lumens consisting of a hollow, elongate, thin-walled outer sheath. The sheath may be constructed from polymer, e.g., at least at a distal part thereof for enhanced flexibility and can be metal at an adjoining proximal part for added strength. A single central core wire extends through the entire length of the sheath. The outer diameter of the core wire is sized close to the inner diameter of the sheath while allowing for axial sliding, in order to maximize the support to the body portion of the snare device. The distal end of the core wire has a tapered section of reduced diameter or cross section to provide a “guidewire-like” flexibility to the distal portion of the device.

In one embodiment, a second wire of about fifty percent or less (approximately 30 percent in an illustrative embodiment) of the inner diameter of the sheath is shaped to form a snare loop and the two ends are attached to the distal most portion of the central core wire via welding, soldering, or brazing.

In another embodiment, a tool tip for removal of thrombus is provided by joining one or more wires that are shaped to provide a radially expanded structure when deployed from the sheath. Both the snare and tool tip can be termed generally a “capture segment” herein.

In still another embodiment, a tool tip for removal of thrombus is provided by a tool tip or capture segment at the distal end of the sheath and core wire that can be controllably expanded to engage a thrombus and remove the thrombus from the blood vessel.

Coatings can be applied to the outer surfaces of the core assembly and the tube assembly to reduce friction between the core and the tube as well as to enhance movement of the snare and thrombus removal device within a catheter. The entire device, when complete, can be made less than 0.014-inch in diameter, and is capable of being placed directly through a percutaneous transluminal coronary angioplasty (PTCA) balloon catheter or other small diameter (micro)catheter that may already be in place within the patient. Alternatively, the snare or thrombus removal device may be passed through the guiding catheter along side of the balloon or access catheter without the need to remove the prior device, and thus, lose temporary access to the site within the patient.

In use, the loop of the snare device is first withdrawn into the sheath by pulling on the actuating handle. The snare device is then advanced into the balloon or guiding catheter until the distal end of the snare has exited the distal end of the guiding catheter. The snare is then torqued and manipulated into place adjacent to the object to be retrieved. The snare loop is exposed from the tube by pushing the actuating handle forward; and through a combination of advancing, withdrawing, and rotating the entire device, the object is ensnared within the loop. The loop is then retracted back into the tube so that the ensnared object is grasped tightly within the loop and the snare with the object is withdrawn from the patient's body.

In use, the expandable thrombus removal tool tip is first withdrawn into the sheath by pulling on the actuating handle. The device is then advanced into the balloon or guiding catheter until the distal end of the sheath has exited the distal end of the guiding catheter. The sheath is then directed into or through the thrombus so that it exits the opposite, distal side of the thrombus. The tool tip is then exposed from the sheath by pushing the actuating handle forward; and once withdrawn, the radially extended tool tip is moved proximally to engage the thrombus. In a planar configuration the tool tip mainly rests on the thrombus' distal face. In a proximally projected (fish hook) configuration, the hooks embed themselves in the material. The device is withdrawn from the patient's body through the vascular system with the thrombus engaged and dragged proximally by the tool tip.

In use, the controllably expansive thrombus removal tool tip is collapsed by pushing on the actuating handle. The device is then advanced into the balloon or guiding catheter until the distal end of the core wire has reached (exited) the distal end of the thrombus. The tool tip is then expanded by pulling the actuating handle backward; and the radially extended (expanded) tool tip is moved to engage the thrombus. The device is withdrawn from the patient's body through the vascular system with the thrombus engaged by the tool tip.

### BRIEF DESCRIPTION OF THE DRAWINGS

The invention description below refers to the accompanying drawings, of which:

FIG. 1 is a partial side cross section of a small-diameter snare device according to an illustrative embodiment of this invention;

FIG. 2 is a full cross section in the region of the attachment between the loop and core wire, taken along line 2-2 of FIG. 1;

FIG. 3 is a cross section of a snare loop wire according to an alternate embodiment having a braided construction;

FIG. 4 is a partial side cross section of the small-diameter snare device including a manipulator handle assembly attached to the proximal end thereof;

FIG. 5 is a full cross section in the region of the slide actuator of the handle, taken along line 5-5 of FIG. 4;

FIG. 6 is a cross section of a pair of D-shaped loop wire sections adjacent to the region of their connection to the core wire according to an alternate embodiment;

FIG. 7 is a partial side cross section of the distal region of a small-diameter catheter for removal of thrombus and other materials according to an illustrative embodiment of this invention;

FIG. 8 is a perspective view of the distal end of the small diameter catheter of FIG. 7 showing the distal tool deployed;

FIG. 9 is a perspective view of the distal end of the small diameter catheter generally in accordance with FIG. 7 showing a deployed distal tool according to a first alternate embodiment;

FIG. 10 is a perspective view of the distal end of the small diameter catheter generally in accordance with FIG. 7 showing a deployed distal tool according to a second alternate embodiment;

FIG. 11 is a perspective view of the distal end of the small diameter catheter generally in accordance with FIG. 7 showing a deployed distal tool according to a third alternate embodiment;

FIG. 12 is a perspective view of the distal end of the small diameter catheter generally in accordance with FIG. 7 showing a deployed distal tool according to a fourth alternate embodiment;

FIG. 13 is a perspective view of the distal end of the small diameter catheter generally in accordance with FIG. 7 showing a deployed distal tool according to a fifth alternate embodiment;

FIG. 14 is a frontal view of the distal end of the small diameter catheter of FIG. 13;

FIG. 15 is an exposed fragmentary view of a blood vessel with a thrombus showing the insertion thereinto of the distal end of the small diameter catheter of FIG. 7 with distal tool retracted;

FIG. 16 is an exposed fragmentary view of the blood vessel with a thrombus of FIG. 15 showing the deployment of the distal tool following insertion thereinto of the distal end of the small diameter catheter of FIG. 7;

FIG. 17 is an exposed fragmentary view of the blood vessel with a thrombus of FIG. 15 showing the withdrawing of the thrombus while engaged by the deployed distal tool of the small diameter catheter of FIG. 7;

FIG. 18 is an exposed fragmentary view of a blood vessel with a thrombus showing the withdrawing of the thrombus while engaged by the deployed distal tool of the small diameter catheter of the embodiment of FIG. 12;

FIG. 19 is an exposed fragmentary view of a blood vessel with a thrombus showing the withdrawing of the thrombus while engaged by the deployed distal tool of the small diameter catheter of the embodiment of FIG. 13.

FIGS. 20A-B illustrates a controllably expansive tool and small-diameter catheter for removal of thrombus and other materials according to an illustrative embodiment of this invention;

FIGS. 21A-B illustrate an embodiment of the controllably expansive tool according to embodiments of this invention;

FIGS. 22A-B illustrate another embodiment of the controllably expansive tool according to embodiments of this invention;

FIGS. 23A-B illustrate another embodiment of the controllably expansive tool according to embodiments of this invention;

FIGS. 24A-B illustrate another embodiment of the controllably expansive tool according to embodiments of this invention;

FIGS. 25A-C illustrate still another embodiment of the controllably expansive tool according to embodiments of this invention;

FIGS. 26A-D illustrate a blood vessel and removal of a thrombus therein by a controllably expansive tool and small-diameter catheter according to embodiments of this invention;

FIGS. 27A-C illustrate a blood vessel and an alternative removal of a thrombus therein by a controllably expansive tool and small-diameter catheter according to embodiments of this invention; and

FIGS. 28A-D illustrate a blood vessel and still another removal of a thrombus therein by a controllably expansive tool and small-diameter catheter according to embodiments of this invention.

### DETAILED DESCRIPTION OF AN ILLUSTRATIVE EMBODIMENT

A. Small Diameter Snare Device and General Design Details

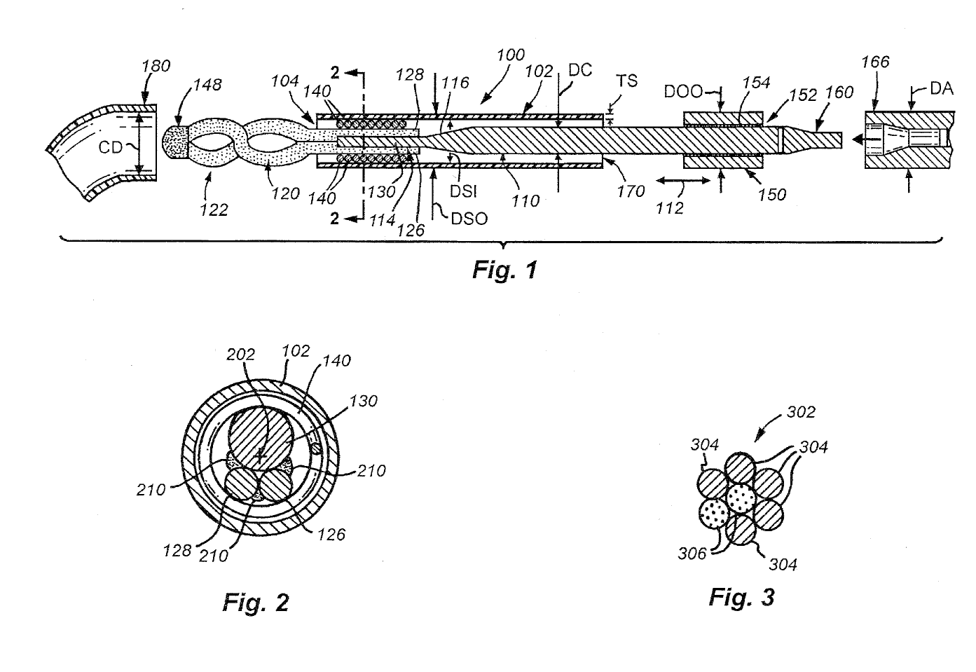

FIG. 1 shows a small diameter snare device 100 according to an embodiment of this invention. Illustratively, the device 100 includes of a hollow, elongate, thin-walled polymer outer sheath 102. The sheath 102 may include a radiopaque marker located at or adjacent to the open distal end 104 for visualization under fluoroscopy. The polymer can be any one of a number of acceptable biocompatible polymers with sufficient structural strength to support a thin-walled (approximately 0.0020 inch maximum wall thickness TS) structure without rupture or other failure under normal use conditions. Alternatively or in addition, the thin-walled outer sheath 102 may be made from a metal tube, a metal spring coil with an outer polymer jacket, or a combination of a metal tube proximal portion and a thin-walled polymer tube distal portion (described below).

In one embodiment, the sheath is constructed from polyimide with a tungsten filler for radiopacity. The radiopaque filler may be added to the sheath polymer during processing, or a radiopaque material may be added to the outer surface via vapor deposition, plating, ion implantation processes, or the like. Alternatively, radiopaque markers can be applied at the distal end and/or other known locations along the sheath, and thus, an overall tungsten filler/radiopaque coating can be omitted. As discussed further below, the outer surface can include thereon a polytetrafluoroethylene (PTFE or “Teflon”) coating upon some, or all, of its outer surface for enhanced lubricity. Alternatively, the outer sheath coating can be constructed form a hydrophilic material that provides lubricity, instead of a PTFE coating. The sheath polyimide material is commercially available for a variety of vendors and sources and is becoming accepted in a variety of medical device applications. It has the property of allowing a very strong, thin-walled cylindrical-cross section tube to be made therefrom, with wall thicknesses on the order of approximately 0.00075 inch to 0.010 inch in normal applications. Nevertheless, the resulting polyimide tube can withstand high pressures in excess of 750 PSI when employed in the size range of the sheath of this invention. Polyimide also resists high temperatures, as much as 1000 degrees F., or greater. Accordingly, polyimide is desirable as a sheath material based upon all of the above-described superior performance characteristics. Nevertheless, it is expressly contemplated that other equivalent plastic/polymer materials suitable for forming a thin-walled sheath tube with similar or better properties (e.g. high strength, thin wall-thickness limits, small diametric sizing) may also be employed as an acceptable “polymer” herein.

The outer sheath 102, which forms the main support and outer framework of the device 100 has an overall length sufficient to traverse the body's varied vasculature, and is (for most applications) permissibly in a range of between approximately 20 cm and 500 cm (more typically between 120 cm and 300 cm). The outer diameter DSO of the sheath is permissibly (for most applications) in a range of between approximately 0.010 inch to 0.045 inch (more typically between 0.010 inch and 0.021 inch), although may fall within the range of 0.008 inch to 0.250 inch diameter. In general, where the outer diameter is less than 0.35 inch, the device 100 may fit easily through a standard balloon catheter.

A single central core wire 110 extends through the entire length of the sheath 102. The outer diameter DC of the core wire through most of the length of the sheath 102 (except near the distal end 104) is sized close to the inner diameter DSI of the sheath while allowing for axial sliding (double arrow 112), in order to maximize the support imparted by the core wire 110 to the body portion/sheath 102 of the snare device 100. (For instance, example guidewire dimensions are 0.014 inch and 0.035 inch diameters.) The distal end 114 of the core wire 110 may have a tapered section 116 of reduced diameter or cross section to provide a “guidewire-like” flexibility to the distal portion of the device. In one embodiment, a second (typically metal) wire 120 of about 50%-30% the inner diameter of the sheath is shaped to form a snare loop 122, and the two ends 126 and 128 are attached to the distal-most portion 130 of the central core wire 110 via welding, soldering, brazing or another high-strength (typical metal-flowing). The loop 122 is typically circular or oval shaped and can also be multiplanar (a twisted “figure-eight” as shown, for example) so as to increase the ability to ensnare and capture objects. Where a multi-planar structure is shown, the entire structure can be referred to collectively as a loop or the two resulting oval perimeters in the figure-eight can be termed in the plural as “loops.” The loop or loops can have a permissible diametric range (their object-grasping inner circumference) of between approximately 1 mm and 100 mm, and typically have a range between 2 mm and 35 mm. However ranges outside the stated values are expressly contemplated.

Note, as used herein, the snare or any other tool tip that selectively extends from the end of the sheath can be termed a “capture segment.”

The central core wire 110 is made from metal for flexibility and strength. In one embodiment, the central core wire 110 may be made by connecting a proximal stainless steel portion, for support and stiffness, to a distal nitinol portion, for torqueability and kink resistance. Likewise, it can be made from 300 series stainless steel or a stronger, heat settable material such as 400 series stainless steel, alloy MP35N, a chromium-cobalt alloy such as Elgiloy, or nitinol in its super elastic or linear elastic state.

Note, because a thin-walled polymer sheath is employed, it advantageously allows for a maximized central core wire diameter, which in turn, provides stiffness for torque control and axial pushability in the body of the snare device.

With reference also to FIG. 2, the central core wire distal-most portion 130 may be offset in one axis relative to the central axis 202 of the sheath 102. This allows mounting of the loop ends 126 and 128 in a most efficient cross-sectional-space-saving manner. Surrounding the distal most portion and two loop ends 126, 128 is a helical wrap of platinum (in this embodiment) wire 140. In one embodiment, the wire has a diameter of approximately 0.001 inch. It is applied to the interconnection between the loop and core wire prior to permanent joining-together of the structure. It thereby secures these components in a tight relationship while solder, weldment, brazing, etc. are applied, reducing the risk of unwanted separation/spreading or sliding of components relative to each other. This further ensures a predictable end diameter for the structure, allowing a tighter wall-thickness tolerance without risk of binding between the core wire assembly and inner wall of the sheath 102. During assembly, solder, etc. passes through small gaps formed between wraps of wire 140 to pool in fillets 210 against the components 126, 128 and 130.

In one embodiment, the snare loop 122 may be made from a 300 series or a heat settable material, such as 400 series stainless steel material, MP35N. Likewise, it may be made from a kink-resistant material, such as chromium-cobalt or nitinol alloy. The snare loop may have an optional radiopaque marker 148 located at the distal-most portion of the loop 122 to aid in fluoroscopic visualization. Alternatively, the snare loop(s) may be formed of a radiopaque material, such as platinum to aid in fluoroscopic visualization. Similarly, the snare loops may have a radiopaque coating applied via vapor deposition, plating, ion implantation processes, or the like, to aid in fluoroscopic visualization, or the snare loop(s) may be covered by a coil (not shown) wound from a radiopaque material, such as platinum to aid in fluoroscopic visualization.

In another embodiment (see FIG. 3), the snare loop(s) may be formed of a wire 302 that defines a plurality of stranded or braided members 304 of an appropriate wire strand material, rather than a single, solid wire as shown above. This stranded or braided wire 302 may (in one or more embodiments) include at least one strand (or multiple strands) 306 of a radiopaque material, such as platinum to aid in fluoroscopic visualization. Alternatively, the snare loop(s) may be formed of a radiopaque material-cored tube, such as a tantalum filled chromium-cobalt material, or platinum filled nitinol material (not shown).

While the snare loops are shown as an independent component attached to a separate core wire end, it is expressly contemplated that the core wire and loops can be a unitary component. For example, in an alternate embodiment (not shown), the snare loops can be made from part of the central core by reducing the diameter of the end of the central core and doubling this free distal end over to form the loop. The free distal end is then joined to the more-proximal part of the narrowed distal end of the core wire. The joint can include wrapping with wire (130 above) and soldering, etc. to construct the finished loop structure.

After assembly of the core wire 110 with appropriate loop(s), and its insertion into the sheath 102, a second short, hollow tube is fitted over the proximal end 152 of the central core wire 110 and attached thereto by a filler or adhesive 154 to provide an actuating handle 150 so as to slideably move the central core wire axially (double arrow 112) within the sheath 102, thus selectively exposing and retracting the snare loop 122 from the open distal end 104 of the sheath 102. In one embodiment, the actuating handle 150 may be sized with an outer diameter DOO similarly (or identically) in outer diameter DSO to the main body of the sheath 102. The exposed proximal end 152 of the core wire 110 may include a narrowed-diameter end 160, with a special connection so that an additional length of wire 166 can be attached to it, thereby extending the overall length of the snare device. This extension has a similarly sized outer diameter DA to that of the handle 150 (DOO) and sheath 102 (DSO). The attachment of this similarly small-diameter extension allows for the exchange of one catheter for another catheter over the body of the snare (and extension). The entire snare device when complete (including the actuating handle 150) can be made less than 0.014 inch in overall outer diameter, and is therefore capable of being placed directly through a PTCA balloon catheter or other small-diameter catheter 180 (FIG. 1), having a sufficiently large inner diameter CD, that may already be in place within the patient (e.g. CD>DSO). Since the actuating handle is equally small in diameter, it also passes through the small-diameter catheter with an extension piece joined behind the handle to the attachment end 160, and thereby allowing the device to be guided even deeper into the patient when needed. The snare may also be passed through the guiding catheter along side of the balloon or access catheter without the need to remove the prior device and, thus, lose temporary access to the site within the patient. For example, the snare may be initially passed through the PTCA balloon catheter, which is already located within the target area. The balloon catheter can then be removed and replaced with a larger-inner diameter catheter to allow removal of the object.

The actuating handle 150 may consist of a metal or a polymer tube. In an alternate embodiment (not shown) the actuating handle may consist of a tube slideable within a second metal tube that is attached to the proximal end 170 of the sheath to maintain an axial orientation between the proximal end of the core wire 102 and sheath, thereby minimizing permanent bending or kinking of the core wire at or near this proximal location.

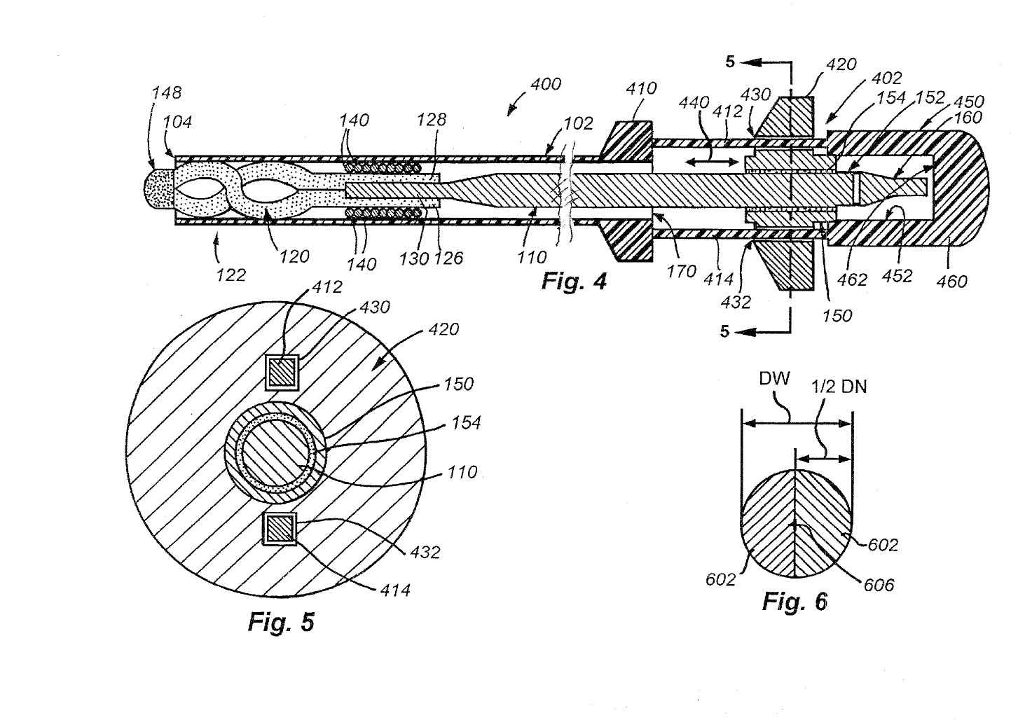

While the depicted actuating handle 150 is of similar outer diameter as the sheath 102, it is expressly contemplated (where the handle will not be passed into another catheter) that the actuating handle may be made in a diameter significantly larger than the snare device so that it may also serve as a torquing handle, similar to those utilized in routine small-diameter guidewire placement. FIG. 4 shows an overall version 400 of the snare device that includes an enlarged handle attachment 402 attachment to the previously described snare device of FIG. 1 (with like components in FIGS. 1 and 4 retaining like reference numbers). The handle attachment 402 may be made from a polymer material which (in an embodiment of this invention) is injection molded and mechanically attached onto the snare or (in another embodiment) may be over molded directly onto the snare. The handle attachment 402 includes a base ring 410 that is secured to the outer surface of the proximal end 170 of the sheath 102. In a detachable-handle embodiment, the ring can consist of a conventional lockable collet structure in which turning of an outer element reduces the diameter of an inner locking element to deliver securing hoop stress to the distal end 170 outer surface of the sheath 102. The base ring is connected to two or more ribs 412 and 414 that are also shown in cross section in FIG. 5.

An actuating ring 420 is secured onto the actuating handle 150 either permanently or detachably. Where it is detachable, it may also utilize a locking collet structure (not shown) as described above. At least two apertures 430 and 432 allow passage of the respective ribs 412 and 414 so that the ring 420, actuating handle 150 and core wire 110 can be slid axially (double arrow 440) with respect to the sheath 102 based upon slideable movement of the actuating ring 420. The ribs secure the ring 420 and interconnected core wire 110 and handle 150 against rotation relative to the sheath. The connection is sufficiently strong so that rotation of the handle assembly 402 causes torquing of the entire device so as to rotate the loop(s) 122 into a desired rotational orientation. In an alternate embodiment, the ring may be a non-circular structure. In another alternate embodiment (also not shown), the ring 420 may also allow at least limited rotation of the core wire relative to the sheath by utilizing arcuate slots at the ribs.

The handle assembly 402 includes a rear gripping member 450. It forms the opposing attachment location for the ribs 412 and 414, opposite the base ring 410. The gripping member can be any acceptable size that provides ergonomic support for a practitioner during a procedure. In one embodiment the member 450 has an outer diameter of approximately ½ to ¾ inch and an external length of approximately 4 to 5 inches. However, it is expressly contemplated that both these dimensions are widely variable outside the stated ranges herein. The member 450 defines an inner cylindrical barrel 452 having an inner diameter sized to slideably receive and guide the proximal end of the actuator handle 150. The barrel 452 has a sufficient length relative to the inner wall 462 of its end cap 460 so that the end 160 of the device does not strike the wall 462 at maximum withdrawal (as approximately shown) of the loop(s) 122 into the sheath 102.

Coatings can be applied to the outer surfaces of the core assembly and the sheath assembly to reduce friction between the core and the tube as well as to enhance movement of the snare device within a catheter. In one embodiment, a lubricious coating, such as PTFE (Teflon), hydrophilic, or diamond-like coating (DLC) may be applied to the outer surface of the sheath to reduce friction. Likewise, one of these coatings may be applied to the outer surface of the core wire to reduce friction with respect to the sheath. Since the coating adds a quantifiable thickness to the thickness of the sheath and/or diameter of the core wire, the overall size of components should be adjusted to compensate for the thickness of any lubricating coating. For example, the outer diameter of the sheath may need to be reduced to maintain a desired 0.035-inch or less outer diameter. Likewise, the thickness of the uncoated wall of the sheath may be reduced to maintain the desired inner diameter and create a final wall thickness, with coating, of approximately 0.0020 inch.

According to an alternate embodiment, as shown in FIG. 6, the loop wire strand 602 (solid in this example) may be made from a half-round or “D-shaped” profile, at least in the vicinity of its joint with the core wire. Note that the advantages of this structure are particularly advantageous in the embodiment described above where the core wire distal end actually forms the loop strand and is joined back on itself so that a separate overlapping core wire end (joined to two separate loop ends) is not present. This D-shaped profile allows for maximizing the cross sectional area of the loop wire thereby increasing its overall breaking strength. For example, a tube with a 0.008-inch inner diameter can accommodate two 0.004-inch diameter round wires stacked together, or the equivalent of a single 0.008-inch (approximately diameter wire if two “D-shaped” wires are stacked. For a given overall desired diameter of DW, the wire strands are half-circular cross sections joined at a line, each having the individual width ½ DW taken through a center point 606 and normal to the joint line between halves. The total cross sectional area of a 0.004-inch diameter wire is 0.000013 square inch, whereas the joined “D-shaped” wire has a cross sectional area of 0.000025 square inch. This results in a doubling of the cross sectional area, and likewise, doubling of the breaking strength of the wire.

Having described the general structure of the snare device and its various alternate embodiments, the operation of the snare device is now briefly described. In use, the loop 122 of the snare device is first withdrawn (proximally) into the sheath 102 by pulling on the actuating handle 150. The snare is then advanced into a balloon or guiding catheter (not shown) until the distal end 104 of the snare device has exited the distal end of the catheter. The snare device is then torqued and manipulated into place adjacent to an object to be retrieved. The snare loop 122 is then exposed (extended) from the open distal end 104 of the sheath 102 by pushing the actuating handle 150 forward (distally), and through a combination of advancing (distally), withdrawing (proximally), and rotating the entire device, the object is ensnared within the loop. The loop is then retracted/withdrawn back into the sheath so that the ensnared object is driven against the distal end 104 of the sheath and grasped tightly within the remaining exposed loop. With the object so-grasped, the snare device with the object is withdrawn (proximally) from the patient's body.

Having described the structure of a snare device according to various embodiments herein and some exemplary techniques for employing the device the following advantages, among others of the above-described invention should be clearer. Namely, this invention provides a small-diameter snare device, less than 0.035 inch in diameter that is capable of fitting through existing balloon or guiding catheters. The body of the snare consists of a thin-walled polymer sheath, which allows for a maximized central core wire diameter, which in turn, provides stiffness for torque control and pushability in the body of the snare device. This device enables addition of one or more extensions onto the proximal end of the snare to allow for exchanging catheters directly over the snare if desired. Portions or all of the sheath and the snare loops can be radiopaque to aid in fluoroscopic visualization. Finally, lubricious coatings can be applied to the outer surface of the core wire and sheath to reduce friction and aid in movement.

B. Expandable Small Diameter Device for Thrombus Removal

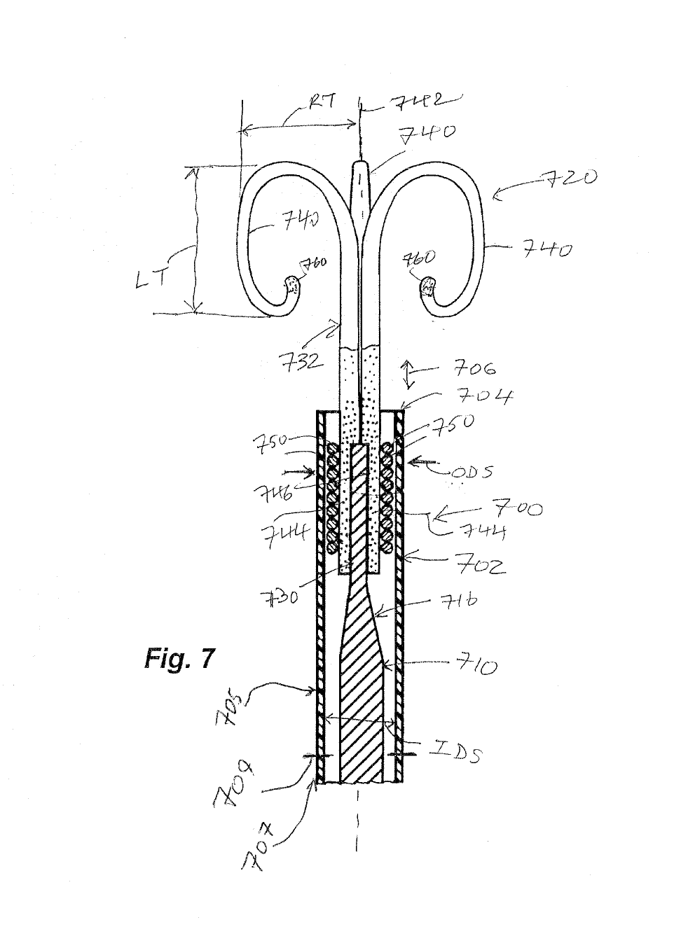

With reference now to FIG. 7, a device 700 for removing thrombus is shown in partial side cross section. The device 700 is a small diameter catheter having a hollow, elongate, thin-walled polymer outer sheath 702 that is substantially similar in materials, structure, and function to the sheath 102 described above for the snare device of FIG. 1. As such, the proximal end (not shown) of the device 700 can be similar or identical in form and structure to the proximal end of the snare device 100 described above. In general, the sheath 702 can be coated with a lubricious coating, such as PTFE, DLC, or hydrophilic polymer material. One material contemplated for the sheath is a polyimide plastic, but a variety of alternate materials displaying the above-described performance characteristics are expressly contemplated. The overall sheath length is in a range of 20 cm to 500 cm (more typically between 120 cm and 300 cm), depending upon (among other factors) the location of the insertion point into the body cavity/vasculature, and the location of the target thrombus, or other material, to be acted upon by the device 700. The outer diameter of the device sheath ODS is in a range of approximately 0.010 inch to 0.045 inch (more typically between 0.010 inch and 0.021 inch). It has a wall thickness of approximately 0.00075 to 0.010 inch in most applications. As described above, a sheath outer diameter less that 0.35 inch enables the device 700 to fit through the lumen of a standard balloon or microcatheter (typically with luminal diameter of 0.014 inch, or less). As described above the core wire's diameter is sized relatively close along the majority of its length (except the distal end) to the inner diameter IDS of the sheath 702.

In this and other embodiments described herein the sheath can be all polymer along its entire length, or can be constructed from a combination of polymer and metal. For example, the distal part 705 of the sheath 702 can be the above-described polyimide material (or another appropriate polymer), while the proximal part 707 can be constructed from 300 series stainless steel or any other appropriate metal. This affords the desired flexibility in the distal part, while providing greater strength and rigidity against buckling in the proximal part. Flexure is required less and beam strength (so as to assist in driving the device distally) is required more in the proximal part 707. The distal part 705, is joined to the proximal part 707 at a joint 709 located at a predetermined distance along the device. The joint 709 can be accomplished using adhesive or any other acceptable joining technique. In one example, the polymer distal part is approximately 40 centimeters in length, while the metal proximal part is approximately 140 centimeters in length. These measurements are widely variable depending upon the overall length of the sheath 702, the purpose of the device (e.g. where it will be inserted) and the distance of the distal part in which high flexibility is required.

The same actuating handle attachment 402 can be employed to move the distal tool 720 of the device 700 into and out of (proximally and distally-double arrow 706) the distal end 704 of the sheath 702.

As with the above-described snare device 100, the device 700 employs a central core wire 710 that moves distally and proximally within the sheath under bias of a handle assembly (handle 402, for example) attached at the sheath's proximal end. The core wire 710 can be constructed from 300 series stainless steel or a stronger, heat settable material, such as 400 series stainless steel, alloy MP35N, a chromium-cobalt alloy, such as Elgiloy, or nitinol in its super elastic or linear elastic shape. Like that of the snare device, the core wire 710 can be constructed by connecting a proximal stainless steel portion, for support and stiffness, to a distal nitinol portion, for torqueability and kink resistance.

The distal region of the core wire 710 is defined by a tapered section 716 as described above. The tapered section necks to a reduced diameter, generally cylindrical distal most portion 730. This narrowed distal area of the core wire 710 affords the above-described guidewire-like flexibility to the distal portion of the device 700. The dimensions for each wired portion can be similar or identical to those described above for the core wire 110.

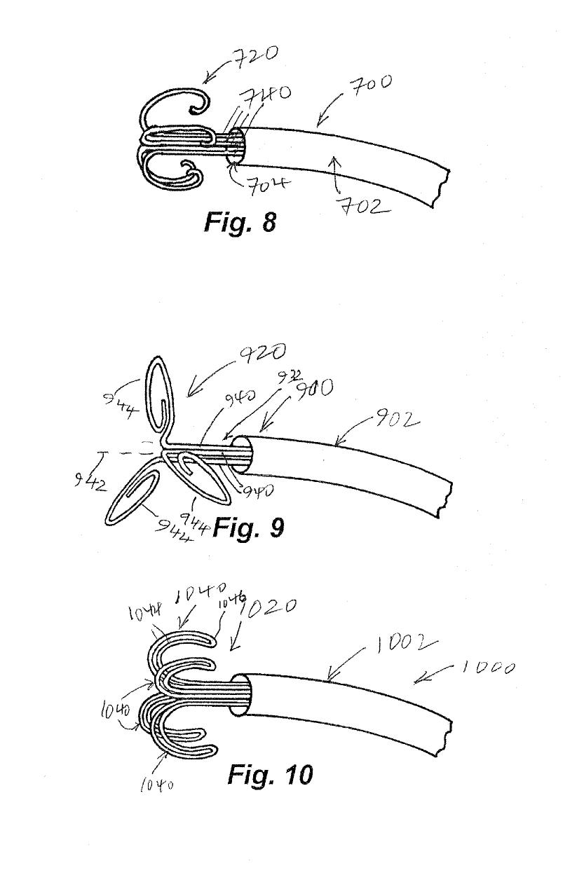

Notably the capture segment in this embodiment is a distal tool tip 720 consisting of a plurality of wires 740 (in this example, four wires at right angles—as shown in FIG. 8), each formed into a predetermined shape. In this embodiment, the shape is an open loop in the form of a “fish hook” that extends distally from the sheath tip 704 in a stalk 732, thereafter opening up and curving radially outwardly from the device center axis 742, and then proximally to end in a radially inward hooked tip. This shape can be loosely termed a “grappling hook” or “umbrella” shape.

Each wire 740 is sized so that the bundle can be drawn inwardly, fully through the distal tip 704 of the sheath 702. The wires 740 are constructed from a metal having substantial flexibility, kink resistance and memory of its shape. Acceptable metals include, but are not limited to, 300 series stainless steel, a heat settable material, such as 400 series stainless steel or another material, such as cobalt-chromium alloy (Elgiloy, nitinol, etc). The wires 740 each include proximal ends 744 that are formed to closely conform to the narrowed distal most portion 730 of the core wire 110. The wires 740 and/or the portion 730 can be formed with flats, half-rounds or steps to more-closely pack the wires 740. In this embodiment, the wires can be offset in accordance with the cross section of FIG. 2. The wires 740 may include a variety of cross sectional shapes, similar to the D-shape shown in FIG. 6. In particular, where four closely conforming wires are employed, as in FIGS. 7 and 8, the wire cross sections can be a quarter of a circle (a right-angled pie piece), so that all four wires, when stretched out along the axis 742 and packed together can form an approximate circular cross section. In this embodiment, the wires 740 are secured to the core wire 710, using welds, solder, brazing, adhesives in combination with a wrapped, helical coil of platinum wire 750 as shown and also described above (refer to wire 140 in FIG. 1). The coil wire 750 has a diameter of approximately 0.001 inch in this embodiment. As described above solder can be flowed between the wire gaps during assembly to fill the spaces therebetween, and render the connection strong and permanent.

The range of radial extension of a fully-deployed tool tip or other capture segment is highly variable. It can be anywhere from 1 millimeter to 100 millimeters in various embodiments. This radial sizing depends partly upon the size of the space into which the capture segment is being inserted. More typically, a capture segment will have maximum radial extension between approximately 2 millimeters and 35 millimeters.

As described above one or more of the wires 770 can include an applied radiopaque marker (tungsten, for example) 760 at any location or a plurality of locations thereon. In this example a marker 760 is applied to each wire tip. The projecting distal-to-proximal length LT is highly variable. In other embodiments described herein below, the distal-to proximal projection can be approximately zero. The dimension should be sufficient in particular designs to ensure appropriate grasping and capture of a thrombus (described further below). The radial projection RT of each wire 740 or the distal tool tip 720 as a whole from the axis 742 should be sufficient to cover the approximate dimension of the cross section to be cleared, while remain smaller than the inner lumen of any vessel through which the deployed tool is expected to carry. This helps to reduce the chance of injury to vascular walls. Also the packed, axially directed stalk section 732 should be long enough to allow the bundle to fully deploy from the sheath tip 740 and exhibit sufficient space from the sheath tip 704 so that the sheath tip does not interfere with the engagement of the tool tip 720 with a thrombus or other target structure.

FIG. 9 details a device tip 900 according to an alternate embodiment. The sheath 902 is constructed similarly to that of FIGS. 7 and 1. Likewise the internal core wire (not shown, is also constructed in accordance with the above described embodiment (FIGS. 1 and 7). The tool tip in this embodiment is constructed from three wires 940. They can be joined to the core wire's narrowed distal most portion using helical wraps and solder as described above. The proximal stalk can be closely packed and each wire can define a cross section that, when extended along the central axis, defines a complete circle with each wire 940 defining a third-of-a-circle pie piece (120 degrees) in this example. Note that in any of the embodiments herein the tool tip wires can alternatively define a circular cross section or another regular polygon. In the embodiment of FIG. 9, the three wires 940 define loops that extend radially outward in a narrowed spiral, and lie substantially within a plane perpendicular to the central axis 942. This arrangement acts collectively as a perforated plate member that is deployed ahead of a thrombus and then engages it from behind. This activity is described more fully below.

FIG. 10 is a device 1000 according to another alternate embodiment. This shape is a version of the “grappling hook” or “umbrella” design that employs loops of continuous wire to define each separate hook member. This device comprises a sheath 1002 also similar to those described above. The tool tip 1020 projects from the sheath distal end to define four wire assemblies. In this embodiment, the wire assemblies 1040 extend radially and proximally, similarly to the embodiment of FIG. 8. These wire assemblies 1040 are formed as narrow pairs of wires 1044 that terminate proximally in a curved joint 1046. This multi-wire arrangement affords greater surface area to the overall tool, which is helpful in ensuring an appropriate grasp on a thrombus or other material. The curved joint ends 1046 also blunt the leading edges of the tool, which engage the material, reducing the chance of the tool passing proximally through the material and also puncturing or scraping vascular walls. The individual wires (eight wires total) of the proximal stalk 1032 are arranged to surround the core wire in a manner that allows the entire tool tip to move into and out of the sheath 1002. The wires can have a cross section shape along all or part of their length, which better facilitates this packing (e.g. ledges, pie piece shapes, etc.).

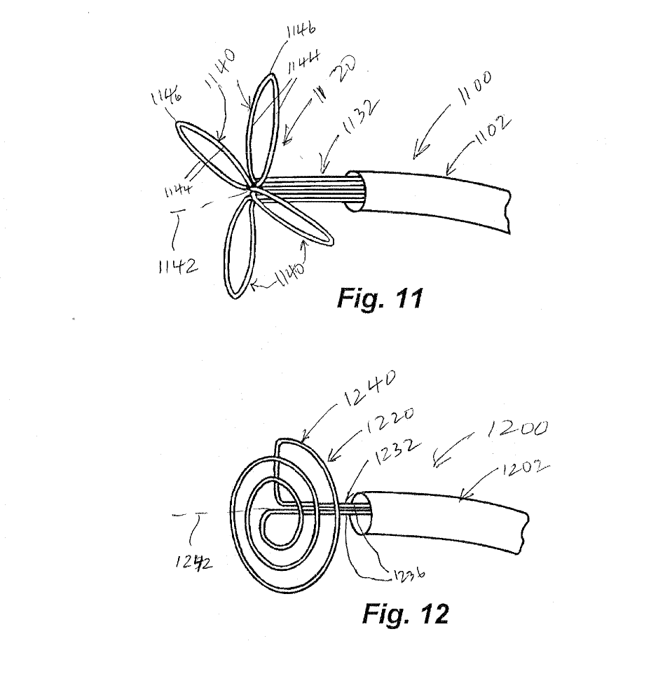

FIG. 11 details a device 1100 according to another alternate embodiment in which the tool tip 1120 that exits the distal end of the sheath 1102 includes four looped wire assemblies 1140 similar to the embodiment of FIG. 10 that essentially define a “flower petal” shape. These wire assemblies extend from a proximal stalk of eight wires, like that of FIG. 10. The distal ends of the wire assemblies 1140 are arranged substantially in a plane, generally perpendicular to the axis 1142 of the sheath distal end. This and other tool tips herein are said to lie substantially within a plane because it is recognized that wires must often overlap each other as they are formed into the desired shape, thereby taking then slightly out of a common plane. The wire assemblies 1140 comprise a pair of wires 1144 that are joined at a radially outermost corner 1146. The wires 1144 tend to bow away from each other between the corners 1146 and the central stalk 1132. This stalk 1132 comprises eight wire ends that are all secured to the core wire in a manner described above (refer to FIG. 10 description).

FIG. 12 defines a device with a sheath 1202 and tool tip 1220. This tool tip defines, in essence a “potato masher” shape employing one continuous wire 1240 that starts and ends in the proximal stalk 1232. The stalk, thus, contains only two wire shafts 1236 joined to the core wire, similar in interconnection to the snare device of FIG. 1. The wire 1249 is formed into a multi-layer spiral as shown. The spiral all lies substantially in a plane perpendicular to the sheath distal end axis 1242. The Spiral affords a high degree of surface area for engaging a thrombus or other material. It also defines an approximately circular outer perimeter that better conforms to the shape of a vascular lumen. Likewise, this outer perimeter shape is less likely to scrape or damage the vascular wall. As in any of the tool tip designs described herein, the maximum outer diameter/radial extension of then tip is selected to conform to the luminal dimensional of the blood vessel in which the tip is deployed.

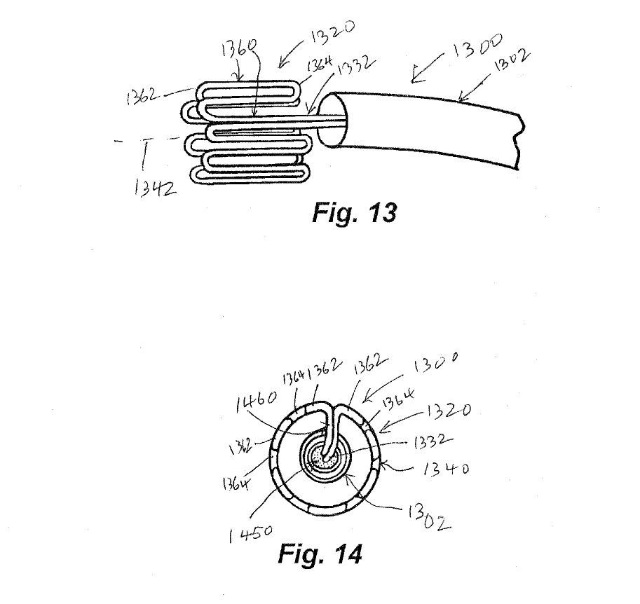

Another embodiment of a device 1300 is detailed in FIGS. 13 and 14. The device 1300 includes a sheath 1302 for which is deployed a tool tip 1320 that, like the tip of FIG. 12, is constructed from a continuous wire 1340 that begins and ends at the core wire (1450 in FIG. 14) within the sheath 1302. These wire ends can be connected to the core wire in the manner described above (refer to description of FIGS. 1 and 12). The stalk 1332 of the tool tip 1320 extends along the distal end axis 1342 of the device 1300, and then deviates radially as shown in FIG. 14 in a connecting wire section 1460. The wire 1320 extends from the connecting section 1460 in a series of proximally directed undulations 1360. The undulations extend in a distal-to-proximal direction and are joined by alternating distal and proximal bend joints 1362 and 1364, respectively. The overall tool tip structure defines a circle around the sheath 1302 and stalk 1332 that is like a cylindrical cage.

Note that each of the tool designs described herein is by way of example. These designs represent various classes of designs that can be employed. Some tool tips employ a continuous wire (FIGS. 12-14). Some reside wire arrangements substantially within a single perpendicular plane (FIGS. 9, 11 and 13). Some terminate in free end tips (FIGS. 8 and 9). Some extend both radially and proximally (FIGS. 8, 10 and 13-14). Some employ a series of continuous wires having bent tip (FIGS. 10 and 11). Some are combinations of these alternative features. The size of the loops, bends and spirals can be varies based upon the size of the target vessel, as well as the size and characteristics of the material being engaged.

As in the snare device, each discrete “wire” in the tool tips described herein can be composed of a plurality of individual strands, collectively defining a cable. One or more strands can be radiopaque (refer to the description of FIG. 3). Notably, the capture segments described herein can be formed in a variety of ways with markers as appropriate. In one example, the capture segment can be formed of a stranded or braided material, with multiple strands of kink-resistant material and at least one strand of a radiopaque material, such as platinum. Alternatively, the capture segment can be formed from a radiopaque-cored tube, such as tantalum filled with chromium-cobalt material, or platinum-filled nitinol material. In a further alternative, the capture segment may be formed unitarily with the distal end of the core wire, by reducing the diameter of the distal end of the core wire to form an appropriate capture segment within this region.

C. Procedures for Withdrawal of Thrombus and Other Materials with Expanding Capture Segments

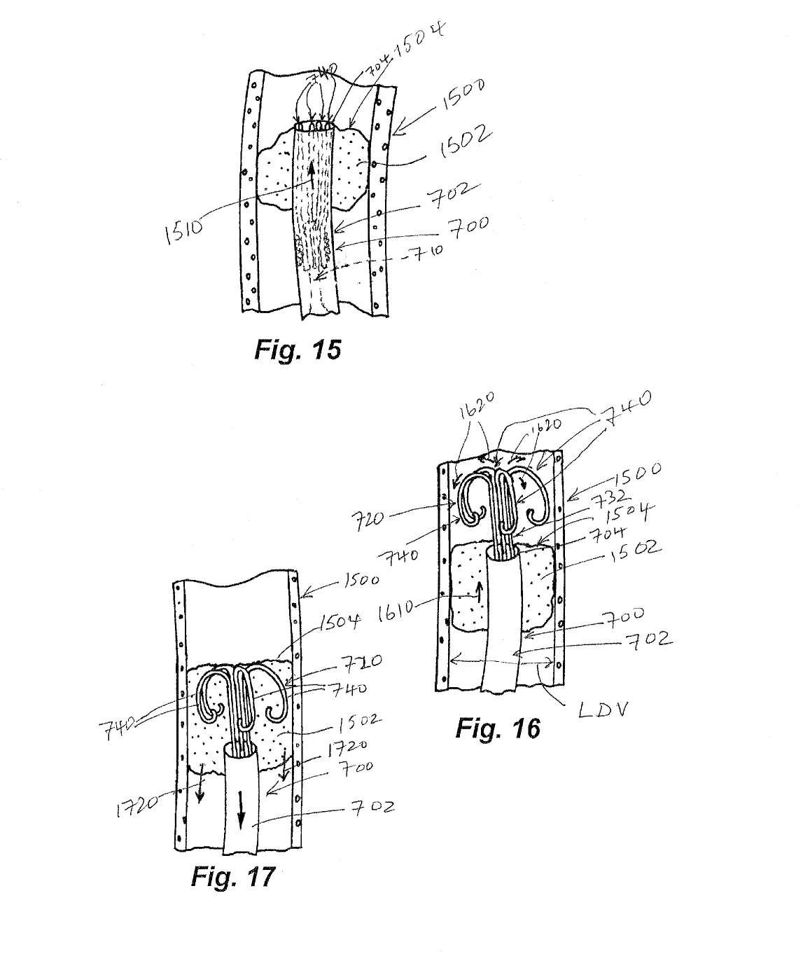

Having described a number of different tool tip designs, the procedure of removing a thrombus or other material from a blood vessel is now described in further detail. FIG. 15 shows the insertion of the device 700 of the embodiment of FIG. 7 into a blood vessel 1500 containing a thrombus, or other internal blockage (natural or man-made). Initial insertion can be made via the aorta or another great blood vessel. The thrombus material is sufficiently accretive to itself so that it can be grasped and withdrawn without fragmentation—which could lead to a thromboembolism or another undesirable effect. As shown, the individual wires 740 (shown partially in phantom) are fully withdrawn/retracted proximally into the distal end of the sheath 702. The wires 740 have sufficient resilience/flexibility to be forcibly drawn into the sheath, and out of their normal hooked, proximally directed shape due to the nature of the material from which they are constructed. As such they are arrayed in a proximal-to-distal alignment against the restraining force of the inner luminal wall of the sheath. Note that the wires are anchored to the core wire 710 in a manner generally described above.

In accordance with FIG. 15, the sheath 702 is directed distally (arrow 1510) so as to pierce fully through the thrombus 1502 as shown, exiting the distal side 1504 of the thrombus 1502. The distal tip 704 is sufficiently small in diameter so that it can pierce the thrombus without significant effort. In alternate embodiment, the tip 704 can contain another chamfer so as to form a piercing chisel point, or another structure that facilitates piercing of the thrombus.

Once pierced, the handle (refer above to the description of FIG. 4) is actuated to drive the core wire 710 distally (arrow 1610) so that the wires 740 are directed out (arrows 1620) of the distal tip 704 of the sheath 702. The memory properties of the wire material cause them to each assume the preformed, proximally and radially directed hook shape of the deployed tool tip 720 as shown. At this time the stalk causes the tool tip 720 to reside at a distance distally spaced from the distal face 1504 of the thrombus 1502. As such, the tool tip 720 can fully deploy without interference from the thrombus 1502. In this embodiment, the radial extension of the wires 740 is slightly smaller than the luminal diameter LDV of the vessel 1500 so that the tool tip 720 can fully deploy without engaging or scraping the vessel wall.

Once deployed, the position of the core wire 710 with respect to the sheath 702 can be locked using, for example the above-described handle lock mechanism. The device 700 is then drawn proximally (arrow 1710) until the tool tip 720 begins to implant itself into the distal face 1504 of the thrombus 1502. The wires 740 are shaped so that they flex appropriately upon engagement with the thrombus 1502, thereby preventing them from passing proximally fully through the thrombus 1502. As such all or a large portion of the thrombus is captured and can be withdrawn proximally (arrows 1720) with the device 700. The thrombus 1502 is held to the tip 720 due to the embedding of the wires 740 so that the thrombus remains intact as it is passed out of the vessel 1500, and into wider diameter blood vessels as it is directed out of the body via the device's point of entry (typically a major vein).

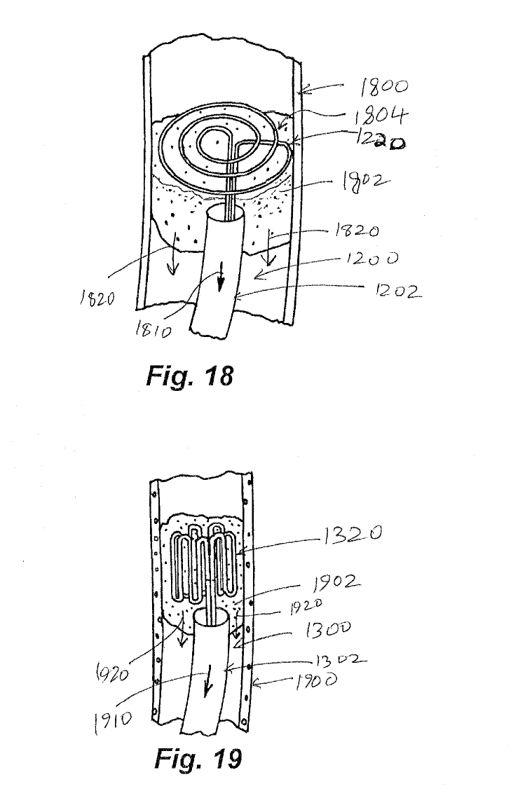

The tool tip 720, and associated procedure described in FIGS. 15-17 entails the embedding of the tool tip into the thrombus upon withdrawal. In alternate embodiments, the tool tip may not be particularly embedded during withdrawal. Reference is made to FIG. 18 in which the device 1200 (FIG. 12) has been driven distally into a vessel 1800 and through a thrombus 1802 in a manner described with reference to FIGS. 15 and 16. The tool tip 1220 has now been deployed from the distal end of the sheath 1202, and taken its preformed shape. The wide and planar shape of the tip 1220 causes it to rest upon the distal face 1804 of the thrombus 1802 without passing substantially into the material. As shown, when withdrawing the device 1200 proximally (arrow 1810), the engaged tool tip 1220 bears forcibly against the distal face 1504 of the thrombus 1802, and withdraws it proximally (arrows 1820).

Referring now to FIG. 19, the sheath 1302 of the device 1300 (FIG. 13) has been driven at least part of the way through a thrombus 1902 in the vessel 1900. The distal-to proximal undulating “cage” design of this tool tip 1320 makes it possible to deploy from inside the thrombus 1902. This entails the cage shape being radially compressed along its undulations in its retracted state and thereafter expanding radially when freed from the end of the sheath. The expansion occurs within the thrombus body, thereby anchoring the tool tip 1320 within its center as shown. Alternatively, this cage-shaped tool tip 1320 can be deployed distally of the distal thrombus face 1904 in a manner described above. After either type of deployment, the device 1300 is withdrawn proximally (arrow 1910), thereby pulling the anchored thrombus 1902 along with it.

D. Controllably Expansive Small Diameter Device for Thrombus Removal

In addition to the expanding capture segments described above, one or more embodiments of the present invention provide for controllably expansive capture segments that reside beyond the outer sheath. In a collapsed state, the OD of the capture segments thus need not fit within the ID of the outer sheath, but may illustratively be sized similar to the OD of outer sheath itself (e.g., no greater than an ID of a catheter in which the outer sheath/device is meant to traverse). In a capture (expanded) state, the capture segments extend to approximately the vessel diameter so that they may be used to capture a thrombus or other material within the vessel and thus move the thrombus/material, e.g., removing it from the vessel or otherwise repositioning the thrombus/material to another location.

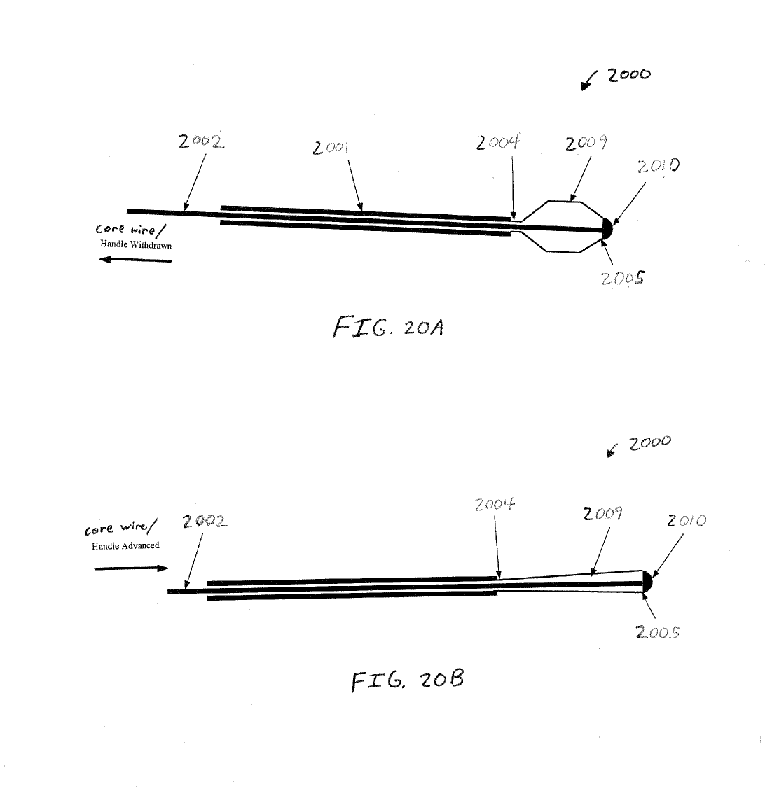

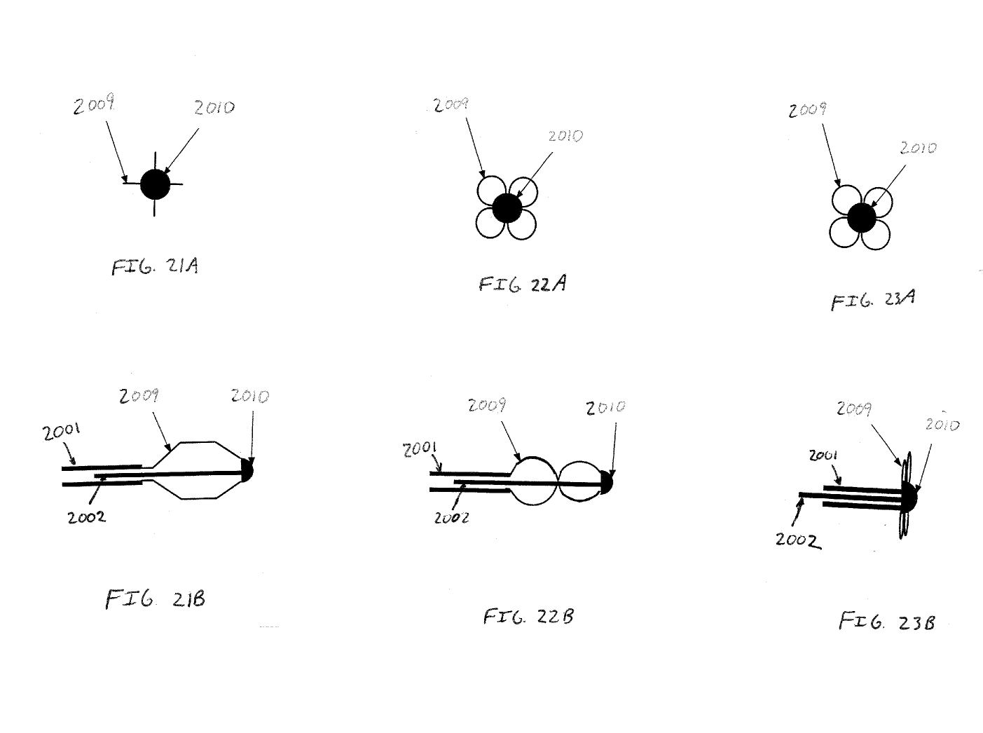

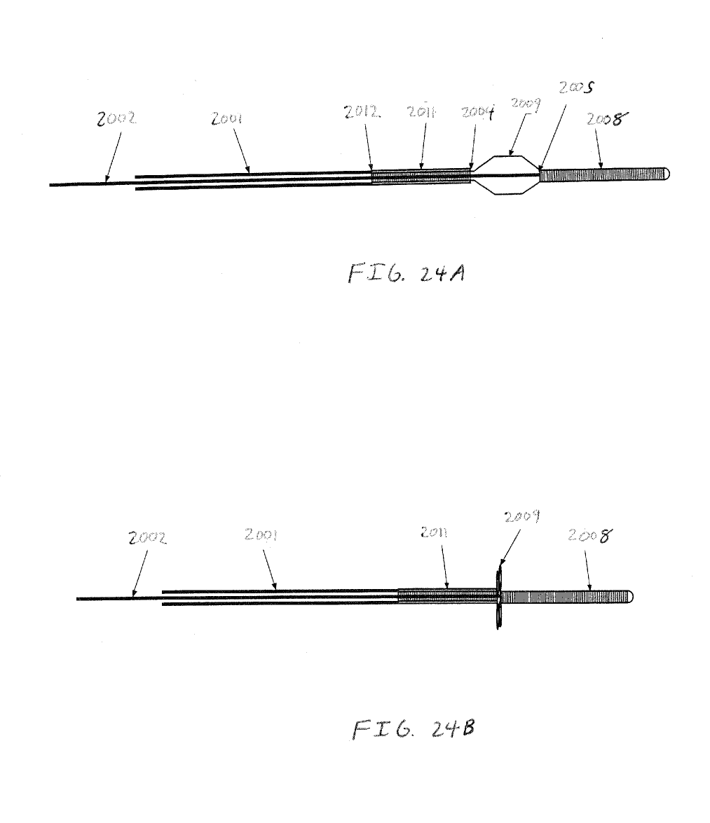

For example, FIGS. 20A and 20B show one embodiment of a capture segment for a thrombus retrieval device that is controllably expansive. In this embodiment, the capture segment/device 2000 is made from one or more small wires 2009, which may be pre-bent and/or heat set into a desired three-dimensional shape. The wires may be made from nitinol, stainless steel, or cobalt-chromium alloy and may be covered with a radiopaque coating or coil for visibility under fluoroscopy, as mentioned above. The proximal ends of the respective wires 2009 are attached (e.g., welded/soldered/glued/etc.) to the outer sheath 2001 at the distal-most end 2004 thereof. The distal ends 2005 of the respective wires 2009 are attached to the actuating or core wire 2002 at a distal cap 2010. The distal cap 2010 may be rounded to form an atraumatic leading edge to facilitate movement through the blood vessels without causing damage.

For controlling expandability operation of this embodiment, when the actuating/core wire 2002 is advanced forward, as shown in FIG. 20B, the capturing wires 2009 collapse downward against the core wire (a “collapsed state”) so that the device can be introduced into the body/vessel (e.g., with an outer diameter substantially close to that of the outer sheath 2001) and directed to the thrombus location. When the core wire 2002 is withdrawn (as in FIG. 20A), the capturing wires 2009, attached or otherwise restricted from entering the outer sheath 2001, expand outwardly (an “expanded state”) to the desired shape allowing for capture of a thrombus (as described further below). Various techniques may be used to lock or otherwise secure the capture segment in either the expanded or collapsed state, for instance, to allow an operator of the device to securely position the capture segment in one or the other state without having to manually apply consistent force (tension) to the actuating/core wire.

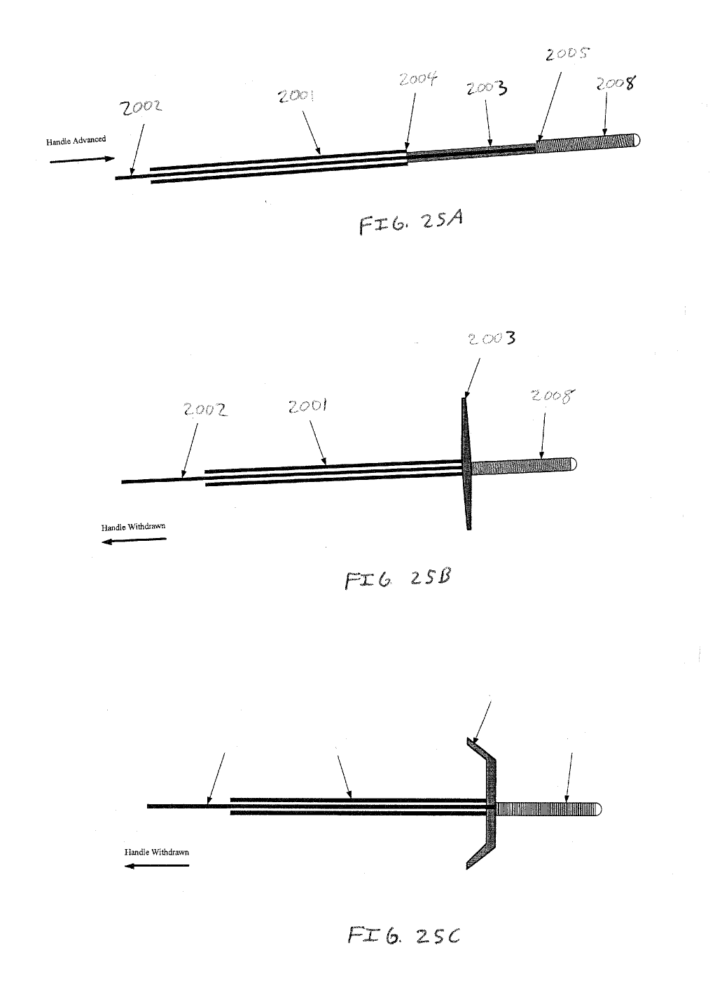

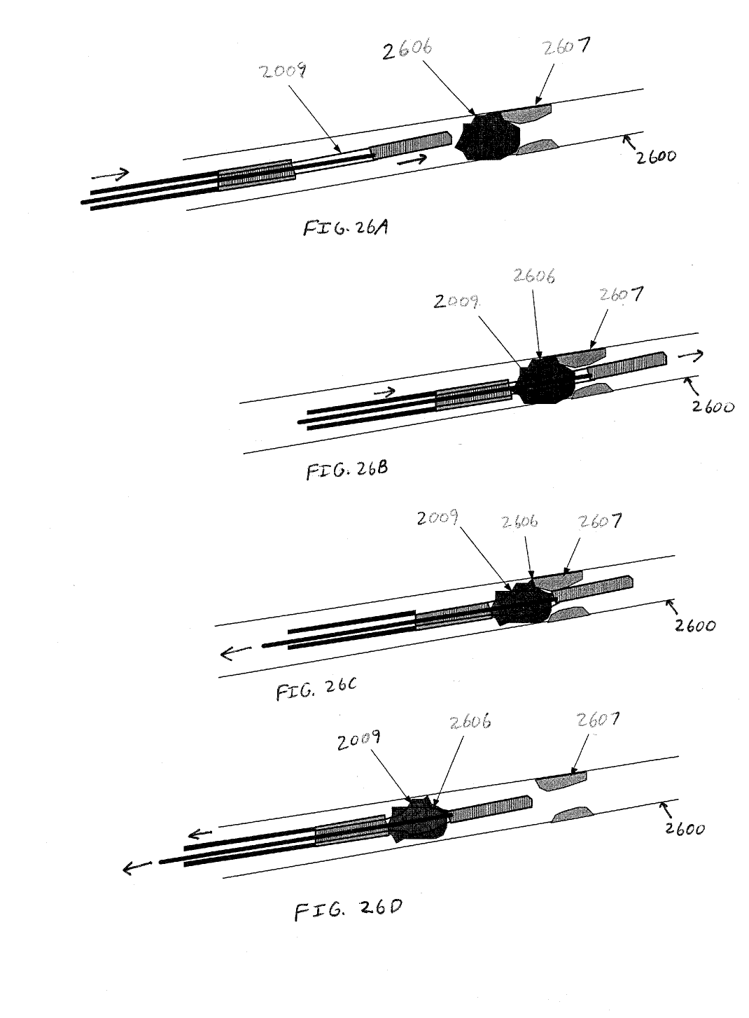

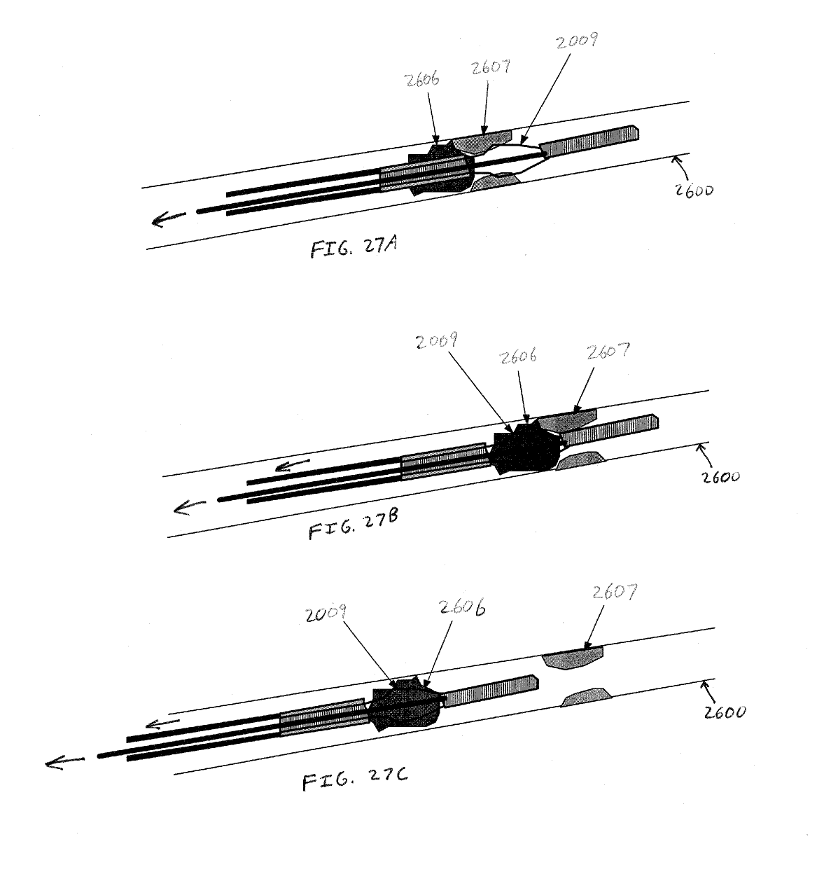

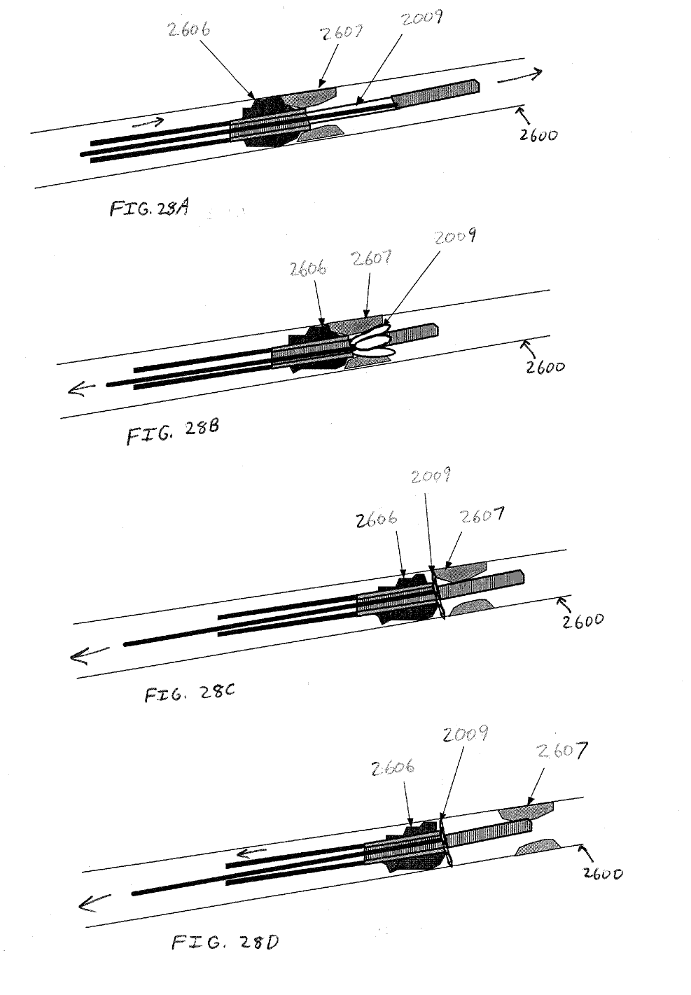

The capturing wires 2009 may be shaped in a single outward plane, such as shown in FIGS. 21A-B, illustrating a cross sectional view (FIG. 21A) and an endpoint view (FIG. 21B) of the capture segment. Alternatively, the wires 2009 may be multi-planar, helical, or looped, such as shown in FIGS. 22A-B, or they may be made into a flattened configuration (e.g., a “flower petal” configuration) as shown in FIG. 23A-B (notably, “flattened” need not imply a two-dimensional shape, but may also comprise becoming reversely concave about the outer sheath 2001 to assist in capturing a thrombus in a “cup-like” manner). In each embodiment, however, the wires 2009 of the capture segment are straightened out and thus collapsed for insertion of the device into the body, when the actuating core wire 2002 is advanced, and expanded (e.g., enlarged and/or flattened) when the core wire is retracted/withdrawn. Further, while the capturing segments are illustratively shown as a symmetric design, the segments (wires 2009) may be configured in multiple fixed diameters, or as other variable/adjustable diameter device not explicitly shown. In use, the actuating wire is withdrawn and locked in position as described above, such that the capture segment remains in the capture/expanded state to remove the thrombus or move the thrombus to a desired location.

In alternative or additional embodiments, the outer sheath 2001 may contain a flexible coil portion on its distal end. For example, FIG. 24A shows a further embodiment of a thrombus retrieval device in which the outer sheath 2001 has a hollow flexible coil segment 2011 added onto its distal end. The coil 2011 provides a flexibility at the distal end of the outer sheath that aids in negotiation of the device through constricted portions of the anatomy, such as that found in the brain (e.g., often extremely tortuous). Coil 2011 is illustratively attached to the distal end of the outer sheath 2001 at connection 2012 in a manner that the ID of the coil remains open to allow the actuating/core wire 2002 to slide therein. In doing so, coil 2011 may be considered as an extension of the outer sheath 1501. In this embodiment, the capture segment wires 2009 are attached at their proximal ends 2004 to the coil 2011 and at their distal ends 2005 to the core wire 2002.

Further, in addition or in the alternative, a distal atraumatic spring portion 2008 may be added to the distal end of the core wire 2002 to facilitate movement through the blood vessels without causing damage. In particular, the core wire 2002 may illustratively continue beyond the distal end of the capture segment (e.g., by approximately 1-3 cm), and may taper to a smaller (e.g., more flexible or “softer”) diameter. A radiopaque spring coil 2008 fits over the end of the core wire and is secured at its distal end to the distal most portion of the core wire. The proximal end of spring coil 2008 is secured to the distal ends of the capturing wires 2009, and the proximate end of the extended core wire.

Briefly, FIG. 24B shows an embodiment of the device after the core wire 2002 has been withdrawn, resulting in expansion of the capture wires 2009 into the illustrative flattened flower petal shape as shown in FIG. 23A-B. As discussed, the core wire is locked at its withdrawn position, and the capture segment remains in its expanded state to capture and remove the thrombus to a desired location.

Notably, while the controllably expansive capture segments described above comprise capture wires 2009, alternative embodiments of the present invention may also utilize a mesh/screen type of material. For instance, as shown in FIG. 25A, the device comprises an outer sheath 2001, a core/actuating wire 2002, and a capture segment (screen) 2003 formed from a braided, metallic material. For example, suitable materials for screen 2003 may comprise nitinol, stainless steel, or cobalt-chromium alloy, although it is conceivable that the braid/screen could also be made from a non-metallic material such as a cloth or polymer fibers. Similar to capture wires 2009 above, the proximal end of the screen 2003 is attached about its periphery to the distal end of the outer sheath 2001, at point 2004, and the distal end of the screen is attached about its periphery to the distal end of the core wire 2002 (and/or the distal coil 2008 noted above) at point 2005.

In operation, as the core/actuating wire 2002 is advanced forward, as shown in FIG. 25A, the braided/screen material is stretched longitudinally causing the body of the braided section to collapse downward against the core wire, as mentioned above with regard to the capture wires 2009. When the core wire 2002 is withdrawn backward, as shown in FIG. 25B, the screen 2003 expands radially as it compresses longitudinally, e.g., into a disc-shaped configuration. Alternatively, as shown in FIG. 25C, the mesh/screen material may also be pre-formed, such by heat setting or other process (e.g., pre-bending), to create a desired shape when compressed to optimize capturing ability, such as the “cup” shape as shown in FIG. 25C. Note that a portion of or the entire mesh/screen segment 2003 may be made radiopaque, such as by adding marker bands, electroplating, or ion-beam bombardment of a radiopaque material onto the mesh. Also, the screen 2003 may be made from a radiopaque material, such as platinum-tungsten wire (e.g., typical of guidewire coils).

Note again that each of the tool designs described herein is by way of example. These designs represent various classes of designs that can be employed. For instance, the size of the capture segments can be varies based upon the size of the target vessel, as well as the size and characteristics of the material being engaged. In addition, while a certain number of capture wires 2009 have been shown (e.g., four wires in FIG. 21A-B, two looping/spiraling wires in 22A-23B) or a number/design of loops (e.g., four loops or “flower petals” of FIG. 22A-23B), these illustrations are merely representative, and should not be limiting on the scope of the present invention.

E. Procedures for Withdrawal of Thrombus and Other Materials with Controllably Expansive Capture Segments

Having described an additional number of different tool tip designs that are controllably expansive, the procedure of removing a thrombus or other material from a blood vessel is again described in further detail with respect specifically to the controllably expansive capture segments/devices 2000. Illustratively, FIGS. 26A-D are a pictorial demonstration of the device from FIG. 24A in use. First, with the core wire advanced forward, thus collapsing the capture segment (capture wires 2009), the device may be advanced into the vessel 2600 and maneuvered into location adjacent to the thrombus 2606 (FIG. 26A). (Notably, the collapsed capture segment should be constructed and assembled in a manner that reduces drag within the vessel or encapsulating catheter, and pre-vents snagging/catching along vessel/catheter walls.)

While the capture segment is maintained in its collapsed state, the device may be pushed through and into the thrombus 2606 (FIG. 26B) so that the capture segment resides at least partially within the thrombus. The core wire 2002 is then withdrawn allowing the capture mechanism to expand outward into the thrombus 2606 (FIG. 26C), thereby containing the thrombus within the capture mechanism. The device is then withdrawn proximally removing the thrombus from the vessel 2600 (e.g., from patient's body) (FIG. 26D). Alternatively, the thrombus 2606 can be retrieved back to a larger area within the body where it can be safely aspirated out of the body, such as by using a large bore catheter and syringe, as will be appreciated by those skilled in the art. Note that the “openness” of various embodiments of the capture segment allows for the segment (spaced wires 2009) to pierce into the thrombus, allowing the thrombus to remain primarily in tact for removal. Conversely, without the openness, penetration into the body of the thrombus by the individual wires 2009 (or mesh 2003) may not only be difficult, but may also result in dilation (or expansion) of the thrombus, and thus adversely affect the removal of the thrombus.

As an alternative to expanding the capture segment within the thrombus 2606, another method of thrombus capture may be used, wherein the capture segment 2000 may be advanced beyond the thrombus 2606 and then opened/expanded as shown in FIG. 27A. The device may then be withdrawn proximally into the thrombus 2600 so that the thrombus becomes contained within the capture segment (FIG. 27B). The device may then be withdrawn proximally removing the thrombus 2606 from the vessel 2600 (FIG. 27C) as described above.

Also, according to one or more embodiments of the present invention, e.g., in accordance with the capture device 2000 with a flattened flower petal shaped capture mechanism 2009 similar to that described in FIG. 23B/24B (or the screen/mesh 2003 of FIGS. 25A-C), FIGS. 28A-D describe another method of thrombus capture. In particular, FIG. 28A shows the device 2000 after it has been pushed through the thrombus 2606 with the core wire 2002 advanced and the capture segment 2009 in its collapsed state, so that the capture segment 2009 (2003) is located beyond the thrombus. In FIG. 28B, the core wire 2002 is withdrawn, allowing the capture segment 2009 (2003) to assume its flower petal (or disc/cup) shape. The device 2000 is then withdrawn as shown in FIG. 28C so that the capture segment comes into contact with the thrombus, thereby dislodging the thrombus from the vessel wall 2600. The device is then withdrawn proximally removing the thrombus 2606 from the vessel 2600 (FIG. 28D). Again, as noted above, the thrombus may be alternatively retrieved back to a larger area within the body where it can be safely aspirated out of the body using a large bore catheter and syringe.

The above-described insertion procedures can be modified to accommodate the characteristics of the particular tool tip shape and size. A variety of additional tools and/or internal scanning devices can be employed to facilitate the procedure in accordance with known medical techniques. In addition, any of the materials or construction techniques described in connection with the thrombus-removal devices herein can be applied to the above-described snare device. In particular, the materials used to form tool tips herein can be used to form the snare. Note also that the proximal end of the thrombus-removal and snare device described herein includes a proximal end that allows removal of the actuator handle and addition of a small-diameter extension. When the extension is added, the practitioner can pass another catheter over the inserted device sheath, thereby using the device as a guide for the larger diameter catheter.

Notably, for each of the expanding capture segments (FIGS. 7-14) and the controllably expansive capture segments (FIGS. 20A-25C) described above should be substantially sized in its expanded state so that it approximates the vessel diameter. For instance, vessel diameters where such a device may be used can typically range from 1 mm to greater than 35 mm, however, most thrombus retrieval procedures are performed in vessels ranging between 1 mm and 10 mm. In this manner, the capture of the thrombus is assisted by substantially preventing the capture segment from simply pulling through the thrombus.

Further, in accordance with one or more embodiments of the present invention, a capture segment may be advantageously coated with a material to attract a thrombus, such as an ionic charge, or may include brushes and/or filaments (not shown). Also, the capture segment may be coated with a thrombus dissolving drug, such as Integrelin®, ReoPro®, or other thrombolytic agents as will be understood by those skilled in the art. Alternatively or in addition, the device may be constructed with a gap between the outer sheath and the actuating/core wire in order that localized drugs (e.g., thrombolytics) may be infused through the outer sheath and delivered directly to the thrombus.

The thrombus removal devices described herein may also operate to open the impeded vessel to allow blood flow. While removal of the thrombus is discussed above, the embodiments may instead be maneuvered within or proximate to the thrombus to puncture and/or break up the thrombus. Also, the thrombolytic agents applied to the capture segment may allow the capture segments to more readily enter/pass through the thrombus.

Moreover, while the above embodiments are described as separate designs and/or aspects, various combinations may also be made to the capturing segments and/or snare. For instance, the controllably expansive capture segments may be originally retracted within the outer sheath as are the expandable capture segments described above. The controllably expansive capture segments may then be released from the outer sheath, and the resultant expansion may be controlled by the core/actuating wire.

The foregoing has been a detailed description of illustrative embodiments of the invention. Various modifications and additions can be made without departing from the spirit and scope of this invention. For example, while specified materials are described, it is expressly contemplated that similar or superior materials may be employed if and when available for the described components of this invention. In particular, a variety of metals, polymers, composite, nano-materials and the like having desirable memory characteristics can be employed for snares, tool tips and other components herein. Likewise, alternate techniques and materials can be employed for joining components. In addition further attachments can be provided to the devices described herein, with appropriate mounting hardware and locations to facilitate other, non-described procedures using the device. Accordingly, this description is meant to be taken only by way of example, and not to otherwise limit the scope of the invention.

What is claimed is:

1. A small-diameter material-removal device, comprising:
a thin-walled outer sheath including a proximal end and a distal end;
a core wire having a proximal end and a distal end, the core wire having an opposing actuator handle at the proximal end that extends from the proximal end of the sheath; and
a capture segment having a proximal end attached to the distal end of the sheath and a distal end attached to the distal end of the core wire, the capture segment having a collapsed state and an expanded state of a predetermined shape, the core wire being constructed and arranged so that applying axial movement to the handle causes the capture segment to controllably expand between the collapsed state and the expanded state for engaging a material within a blood vessel, wherein the capture segment remains in the expanded state for moving the material.

2. The device as in claim 1, wherein the material is a thrombus.

3. The device as in claim 1, wherein the capture segment is a plurality of individual preformed wires.

4. The device as in claim 1, wherein in the collapsed state, the capture segment is sized substantially similar to an outer diameter of the outer sheath.

5. The device as in claim 1, wherein in the expanded state, the capture segment is expanded to approximately an inner diameter of the blood vessel.

6. The device as in claim 1, wherein the predetermined shape of the capture segment is at least one of either pre-bent or heat set.

7. The device as in claim 1, wherein the capture segment comprises at least one of either nitinol, stainless steel, or cobalt-chromium alloy.

8. The device as in claim 1, wherein the capture segment comprises a radiopaque portion that is visible under fluoroscopy.

9. The device as in claim 1, further comprising:
a distal cap having an atraumatic leading edge at the distal end of the core wire.

10. The device as in claim 1, wherein the core wire comprises an extended portion beyond the distal end of the capture segment, the device further comprising:
an atraumatic spring having an atraumatic leading edge on the distal end of the extended portion of the core wire.

11. The device as in claim 1, wherein the distal end of the outer sheath comprises a flexible coil.

12. The device as in claim 1, wherein the capture segment is configured to be secured in one of either the expanded state or collapsed state.

13. The device as in claim 1, wherein the capture segment comprises wires selected from the group consisting of: one or more multi-planar wires; one or more helical wires; and one or more looped wires.

14. The device as in claim 1, wherein the capture segment, in an expanded state, comprises a flower petal configuration.

15. The device as in claim 1, wherein the capture segment further comprises a screen.

16. The device as in claim 15, wherein the screen comprises a material selected from the group consisting of: a braided material; a metallic material; and a non-metallic material.

17. The device as in claim 16, wherein the non-metallic material is selected from cloth and polymer fibers.

18. The device as in claim 15, wherein the screen, in an expanded state, comprises one of either a disc-shaped configuration or a cup-shaped configuration.

19. The device as in claim 1, wherein the capture segment comprises a material to attract a thrombus.

20. The device as in claim 19, wherein the material is selected from a group consisting of: an ionic charge, brushes, and filaments.

21. The device as in claim 1, further comprising:
a thrombus dissolving drug coated on the capture segment.

22. The device as in claim 1, wherein the core wire, outer sheath, and capture segment are configured to allow for infusion of drugs from the proximal end of the outer sheath to the distal end of the outer sheath.

23. A method for use with a small-diameter material-removal device having a thin-walled outer sheath including a proximal end and a distal end, the device further having a core wire having a proximal end and a distal end, the core wire having an opposing actuator handle at the proximal end that extends from the proximal end of the sheath, the device further having a capture segment having a proximal end attached to the distal end of the sheath and a distal end attached to the distal end of the core wire, the capture segment having a collapsed state and an expanded state of a predetermined shape, the method comprising:
applying axial movement to the handle to cause the capture segment to controllably expand between the collapsed state and the expanded state;
penetrating a material within a blood vessel with the capture segment in the collapsed state; and
engaging the material with the capture segment, wherein the capture segment remains in the expanded state to move the material.

24. The method as in claim 23, further comprising:
removing the material from the blood vessel with the capture segment in the expanded state.

25. The method as in claim 23, further comprising:
securing the capture segment in one of either the collapsed state or the expanded state.

26. A method of making small-diameter material-removal device, comprising:
providing a thin-walled outer sheath including a proximal end and a distal end;
inserting a core wire into the outer sheath, the core wire having a proximal end and a distal end, the core wire having an opposing actuator handle at the proximal end that extends from the proximal end of the sheath;
constructing a capture segment having a distal end and a proximal end, the capture segment having a collapsed state and an expanded state of a predetermined shape;
attaching a capture segment at a proximal end to the distal end of the sheath; and
attaching a distal end of the capture segment to the distal end of the core wire, the capture segment and the core wire being constructed and arranged so that applying axial movement to the handle causes the capture segment to controllably expand between the collapsed state and the expanded state.
